(19) United States
(12) Patent Application Publication
(10) Pub. No.: US 2021/0275447 A1
(43) Pub. Date: Sep. 9, 2021

(54) SUSTAINED-RELEASE OPHTHALMIC PHARMACEUTICAL COMPOSITIONS AND USES THEREOF

(71) Applicants: TAIWAN LIPOSOME CO., LTD., Taipei City (TW); TLC BIOPHARMACEUTICALS, INC., South San Francisco, CA (US)

(72) Inventors: Keelung HONG, South San Francisco, CA (US); Hao-Wen KAO, South San Francisco, CA (US); Yi-Yu LIN, South San Francisco, CA (US); Weiwei FANG, South San Francisco, CA (US)

(21) Appl. No.: 17/270,747

(22) PCT Filed: Sep. 9, 2019
(86) PCT No.: PCT/US2019/050135

§ 371 Date: Feb. 23, 2021

Related U.S. Application Data
(60) Provisional application No. 62/729,038, filed on Sep. 10, 2018.

(51) Int. Cl. A61K 9/00; A61K 9/127; A61K 47/26; A61K 47/02; A61K 31/4045; A61K 31/4439; A61K 31/506

(52) U.S. Cl. CPC A61K 9/0048; A61K 9/127; A61K 47/26; A61K 31/506; A61K 31/4045; A61K 31/4439; A61K 47/02

(57) ABSTRACT

The present invention relates to an ophthalmic pharmaceutical composition comprising at least one liposome and a therapeutic agent for treating an eye disease with a high drug to lipid ratio and encapsulation efficiency. Also provided is the method for treating age-related macular degeneration or diabetic eye disease using the ophthalmic pharmaceutical composition disclosed herein.

### CROSS-REFERENCE TO RELATED APPLICATIONS

This application claims the benefit of U.S. Application No. 62/729,038, filed on 10 Sep., 2018, the entire disclosure of which is incorporated herein by reference.

### TECHNICAL FIELD

The present invention is directed to a sustained-release ophthalmic pharmaceutical composition with a high drug to lipid ratio and a high drug encapsulation efficiency using at least one trapping agent. The high drug to lipid ratio, high encapsulation efficiency and sustained release profile of the ophthalmic pharmaceutical composition reduce the frequency of drug administration, increases patient compliance and improves the therapeutic outcome.

### BACKGROUND

Age-related macular degeneration (AMD) is the leading cause of severe vision loss in people aged over 60 years. There are two subtypes of AMD described as either dry or wet. More than 80% of patients have the dry AMD, which may progress to wet AMD and lead to significant vision loss. The pathogenesis of AMD is poorly understood and likely multifactorial, involving genetic defect, oxidative stress, inflammation, lipid and carbohydrate metabolism, and environmental factors. Wet AMD pathology is characterized by the proliferation of blood vessels from the choriocapillaris through Bruch's membrane and into the retinal pigment epithelium and photoreceptor layers. Recent studies have suggested that blocking vascular endothelial growth factor (VEGF) and/or platelet-derived growth factor (PDGF) pathway by intravitreally injecting VEGF receptor tyrosine kinase inhibitors and/or PDGF receptor tyrosine kinase inhibitors is one of the treatment strategies for AMD. It is highly desirable to maintain the therapeutic concentration of the receptor tyrosine kinase inhibitor at the target site and minimize the frequency of intravitreal injection.

Liposomes as a drug delivery system has been widely used for developing sustained-release formulations for various drugs. Drug loading into liposomes can be attained either passively (the drug is encapsulated during liposome formation) or remotely/actively (creating a transmembrane pH- or ion-gradient during liposome formation and then the drug is loaded by the driving force generated from the gradients after liposome formation) (U.S. Pat. Nos. 5,192,549 and 5,939,096). Although the general methods of drug loading into liposomes is well documented in the literature, only a handful of therapeutic agents were loaded into liposomes with high encapsulation efficiency. Various factors can affect the drug to lipid ratio and encapsulation efficiency of liposomes, including but not limited to, the physical and chemical properties of the therapeutic agent, for example, hydrophilic/hydrophobic characteristics, dissociation constant, solubility and partition coefficient, lipid composition, trapping agent, reaction solvent, and particle size (Proc Natl Acad Sci USA. 2014; 111(6): 2283-2288 and Drug Metab Dispos. 2015; 43 (8):1236-45).

There remains an unmet need for a sustained release ophthalmic formulation with a high drug to lipid ratio and high encapsulation efficiency to reduce the frequency of administration and improve therapeutic outcome. The present invention addresses this need and other needs.

### SUMMARY OF THE INVENTION

In one embodiment, a sustained release ophthalmic pharmaceutical composition comprises (a) at least one liposome comprising a bilayer membrane; (b) a trapping agent; and (c) a therapeutic agent for treating an eye disease, wherein the bilayer membrane comprises at least one lipid and the molar ratio of the therapeutic agent to the lipid is equal to or higher than about 0.2 is provided.

According to another embodiment, methods are provided for treating an eye disease, comprising the steps of administering a sustained release ophthalmic pharmaceutical composition described herein to a subject in need thereof. In an exemplary embodiment, the eye disease is AMD or diabetic eye disease.

Also provided are the uses of the sustained release ophthalmic pharmaceutical composition described herein in the manufacture of a medicament for therapeutic and/or prophylactic treatment of an eye disease.

Further provided is a medicament for treating an eye disease, comprising a therapeutically effective amount of the pharmaceutical composition described herein.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

A further understanding of the nature and advantages of the present invention may be realized by reference to the remaining portions of the specification and the drawings.

### BRIEF DESCRIPTION OF THE DRAWINGS

FIG. 1 is a line graph showing the release profile of the free sunitinib and the liposomal sunitinib formulations A and B in the vitreous humor of rabbits.

### DETAILED DESCRIPTION OF THE INVENTION

As employed above and throughout the disclosure, the following terms, unless otherwise herein, the singular forms “a,” “an” and “the” include the plural reference unless the context clearly indicates otherwise.

All numbers herein may be understood as modified by “about.” As used herein, the term “about” refers to a range of ±10% of a specified value.

An “effective amount,” as used herein, refers to a dose of the sustained release ophthalmic pharmaceutical composition to reduce the symptoms and signs of an eye disease (for example, age-related macular degeneration or diabetic eye disease), such as change in visual acuity, dark or blurry areas in the vision, straight lines appearing wavy or distorted, difficulty reading or seeing details in low light levels and extra sensitivity to glare. The term “effective amount” and “therapeutically effective amount” are used interchangeably.

The term “treating,” “treated,” or “treatment,” as used herein, includes preventative (e.g. prophylactic), palliative, and curative methods, uses or results. The terms “treatment” or “treatments” can also refer to compositions or medicaments. Throughout this application, by treating is meant a method of reducing or delaying one or more symptoms or signs of an eye disease (for example, age-related macular degeneration or diabetic eye disease) or the complete amelioration of the eye disease as detected by art-known techniques. Art recognized methods are available to detect age-related macular degeneration or diabetic eye disease and their symptoms. These include, but are not limited to, vision acuity tests, Amsler grid test, dilated eye/fundus examination, optical coherence tomography testing and fluorescein angiogram. For example, a disclosed method is considered to be a treatment if there is about a 1% reduction in one or more symptoms of age-related macular degeneration or diabetic eye disease in a subject when compared to the subject prior to treatment or control subjects. Thus, the reduction can be about a 1, 5, 10, 20, 30, 40, 50, 60, 70, 80, 90, 100%, or any amount of reduction in between.

The term “age-related macular degeneration,” as used herein, encompasses a variety of types and subtypes of age-related macular degeneration of various etiologies and causes, either known or unknown.

The term “diabetic eye disease,” as used herein, encompasses diabetic retinopathy, diabetic macular edema, cataract and glaucoma, or any eye condition caused by diabetes.

The term “subject” can refer to a vertebrate having or at risk of developing an eye disease, including age-related macular degeneration and/or diabetic eye disease or to a vertebrate deemed to be in need of treatment for an eye disease. Subjects include all warm-blooded animals, such as mammals, such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, mouse, rabbit, rat, gerbil, guinea pig, etc.). Thus, veterinary uses and medical formulations are contemplated herein.

### Liposome

The terms “liposome,” “liposomal” and related terms, as used herein, are characterized by an interior aqueous space sequestered from an outer medium by one or more bilayer membranes forming a vesicle. In certain embodiments, the interior aqueous space of the liposome is substantially free of a neutral lipid, such as triglyceride, non-aqueous phase (oil phase), water-oil emulsions or other mixtures containing non-aqueous phase. Non-limiting examples of liposomes include small unilamellar vesicles (SUV), large unilamellar vesicles (LUV), and multi-lamellar vesicles (MLU) with an average diameter ranges from 50-500 nm, 50-450 nm, 50-400 nm, 50-350 nm, 50-300 nm, 50-250 nm, 50-200 nm, 100-500 nm, 100-450 nm, 100-400 nm, 100-350 nm, 100-300 nm, 100-250 nm or 100-200 nm, all of which are capable of passing through sterile filters.

Bilayer membranes of liposomes are typically formed by at least one lipid, i.e. amphiphilic molecules of synthetic or natural origin that comprise spatially separated hydrophobic and hydrophilic domains. Examples of lipid, including but not limited to, dialiphatic chain lipids, such as phospholipids, diglycerides, dialiphatic glycolipids, single lipids such as sphingomyelin and glycosphingolipid, and combinations thereof. Examples of phospholipid according to the present disclosure include, but not limited to, 1,2-dilauroyl-sn-glycero-3-phosphocholine (DLPC), 1,2-dimyristoyl-sn-glycero-3 -phosphocholine (DMPC), 1,2-dipalmitoyl-sn-glycero-3-phosphocholine (DPPC), 1-palmitoyl-2-stearoyl-sn-glycero-3-phosphocholine (PSPC), 1-palmitoyl-2-oleoyl-sn-glycero-3-phosphatidylcholine (POPC), 1,2-distearoyl-sn-glycero-3 -phosphocholine (DSPC), 1,2-dioleoyl-sn-glycero-3-phosphocholine (DOPC), hydrogenated soy phosphatidylcholine (HSPC), 1,2-dimyristoyl-sn-glycero-3-phospho-(1′ -rac-glycerol) (sodium salt) (DMPG), 1,2-dipalmitoyl-sn-glycero-3-phospho-(1′-rac-glycerol) (sodium salt) (DPPG), 1 -palmitoyl-2-stearoyl-sn-glycero-3-phospho-(1′-rac-glycerol) (sodium salt) (PSPG), 1,2-distearoyl-sn-glycero-3-phospho-(1′-rac-glycerol) (sodium salt) (DSPG), 1,2-dioleoyl-sn-glycero-3-phospho-(1′-rac-glycerol) (DOPG), 1,2-dimyristoyl-sn-glycero-3-phospho-L-serine (sodium salt) (DMPS), 1,2-dipalmitoyl-sn-glycero-3-phospho-L-serine (sodium salt) (DPPS), 1,2-distearoyl-sn-glycero-3-phospho-L-serine (sodium salt) (DSPS), 1,2-dioleoyl-sn-glycero-3-phospho-L-serine (DOPS), 1,2-dimyristoyl-sn-glycero-3-phosphate (sodium salt) (DMPA), 1,2-dipalmitoyl-sn-glycero-3-phosphate (sodium salt) (DPPA), 1,2-distearoyl-sn-glycero-3-phosphate (sodium salt) (DSPA), 1,2-dioleoyl-sn-glycero-3-phosphate (sodium salt) (DOPA), 1,2-dipalmitoyl-sn-glycero-3-phosphoethanolamine (DPPE), N-(carbonyl-methoxypolyethyleneglycol)-1,2-dipalmitoyl-sn-glycero-3-phosphoethanolamine (PEG-DPPE), 1-palmitoyl-2-oleoyl-sn-glycero-3-phosphoethanolamine (POPE), 1,2-distearoyl-sn-glycero-3-phosphoethanolamine (DSPE), N-(carbonyl-methoxypolyethyleneglycol)-1,2-distearoyl-sn-glycero-3-phosphoethanolamine (PEG-DSPE), 1,2-dioleoyl-sn-glycero-3-phosphoethanolamine (DOPE), 1,2-dipalmitoyl-sn-glycero-3-phospho-(1′ -myo-inositol) (ammonium salt) (DPPI), 1,2-distearoyl-sn-glycero-3-phosphoinositol (ammonium salt) (DSPI), 1,2-dioleoyl-sn-glycero-3-phospho-(1′-myo-inositol) (ammonium salt) (DOPI), cardiolipin, L-a-phosphatidylcholine (EPC), and L-α-phosphatidylethanolamine (EPE). In some embodiments, the lipid is a lipid mixture of one or more of the foregoing lipids, or mixtures of one or more of the foregoing lipids with one or more other lipids not listed above, membrane stabilizers or antioxidants.

In some embodiments, the mole percent of the lipid in the bilayer membrane is equal or less than about 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45 or any value or range of values therebetween (e.g., about 45-85%, about 45-80%, about 45-75%, about 45-70%, about 50-85%, about 50-80%, about 50-75%, about 50-70%, about 55-85%, about 55-80%, about 55-75% or about 55-70%).

In some embodiments, the lipid of the bilayer membrane is a mixture of a first lipid and a second lipid. In some embodiments, the first lipid is selected from the group consisting essentially of phosphatidylcholine (PC), HSPC, DOPC, POPC, DSPC, DPPC, DMPC, PSPC and combination thereof and the second lipid is selected from the group consisting essentially of a phosphatidylethanolamine, phosphatidylglycerol, PEG-DSPE, DPPG, DOPG and combination thereof. In other embodiments, the mole percent of the first lipid in the bilayer membrane is about 84.9, 84.3, 84.1, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45 or any value or range of values therebetween (e.g., about 45-84.9%, about 45-80%, about 45-75%, about 45-70%, about 50-84.9%, about 50-80%, about 50-75%, about 50-70% or about 55-70%) and the mole percent of the second lipid in the bilayer membrane is between 0.1 to about 20, 19, 18, 17, 16, 15, 14, 13, 12, 11, 10, 9, 8, 7 or any value or range of values therebetween (e.g., about 0.1-20%, about 0.1-15%, about 0.1-10% about 0.5-20%, about 0.5-15%, about 0.5-10% or about 0.5-7%).

The bilayer membrane of the liposome further comprises less than about 55 mole percentage of steroids, preferably cholesterol. In certain embodiments, the mole % of steroid (such as cholesterol) in the bilayer membrane is about 15-55%, about 20-55%, about 25-55%, about 15-50%, about 20-50%, about 25-50%, about 15-45%, about 20-45%, about 25-45%, about 15-40%, about 20-40% or about 25-40%.

In one exemplary embodiment, the mole % of the lipid and cholesterol in the bilayer membrane is about 45-85%: 15-55%, 45-80%: 20-55% or 50-85%:15-50%. In another exemplary embodiment, the mole % of the first lipid, the second lipid and cholesterol in the bilayer membrane is about 45-84.9%: 0.1-20%: 15-55%, 50-80%: 0.1%-20%: 15-50% or 55-75%: 0.5-20%: 20-45%.

### Remote Loading

The term “remote loading,” as used herein, is a drug loading method which involves a procedure to transfer drugs from the external medium across the bilayer membrane of the liposome to the interior aqueous space by a polyatomic ion-gradient. Such gradient is generated by encapsulating at least one polyatomic ion as a trapping agent in the interior aqueous space of the liposome and replacing the outer medium of the liposome with an external medium with a lower polyatomic ion concentration, for example, pure water, sucrose solution and saline, by known techniques, such as column separation, dialysis or centrifugation. A polyatomic ion gradient is created between the interior aqueous space and the external medium of the liposomes to trap the therapeutic agent in the interior aqueous space of the liposomes. Exemplary polyatomic ions as trapping agents include, but are not limited to, sulfate, sulfite, phosphate, hydrogen phosphate, molybdate, carbonate and nitrate. Exemplary trapping agents include, but are not limited to, ammonium sulfate, ammonium phosphate, ammonium molybdate, ammonium sucrose octasulfate, triethylammonium sucrose octasulfate, dextran sulfate, or a combination thereof.

In an embodiment, the concentration of triethylammonium sucrose octasulfate is about 10 to 200 mM, about 50 to about 150 mM. In another embodiment, the concentration of ammonium sulfate is about 100 to 600 mM, about 150 to about 500 mM, about 200 to about 400 mM. In yet another embodiment, the concentration of ammonium phosphate is about 100 to about 600 mM, about 150 to about 500 mM, about 200 to about 400 mM.

In accordance with the invention, the liposome encapsulating a trapping agent can be prepared by any of the techniques now known or subsequently developed. For example, the MLV liposomes can be directly formed by a hydrated lipid film, spray-dried powder or lyophilized cake of selected lipid compositions with trapping agent; the SUV liposomes and LUV liposomes can be sized from MLV liposomes by sonication, homogenization, microfluidization or extrusion.

### Pharmaceutical Compositions

The present invention is directed to a sustained release ophthalmic pharmaceutical composition, comprising (a) at least one liposome comprising a bilayer membrane; (b) a trapping agent; and (c) a therapeutic agent for treating an eye disease, wherein the bilayer membrane comprises at least one lipid and the molar ratio of the therapeutic agent to the lipid is above or equal to 0.2. In some embodiment, the molar ratio of the therapeutic agent to the lipid is above or equal to 0.2 to less than about 20, less than about 15, less about 10, less than about 5.

In one embodiment, the sustained release pharmaceutical composition further comprises at least one pharmaceutically acceptable excipient, diluent, vehicle, carrier, medium for the active ingredient, a preservative, cryoprotectant or a combination thereof. In one exemplary embodiment, the weight percent of the bilayer membrane in the pharmaceutical composition is about 0.1-15%; the weight percent of the trapping agent in the pharmaceutical composition is about 0.1-12%; and the weight percent of the pharmaceutically acceptable excipient (such as sucrose, histidine, sodium chloride and ultrapure water), diluent, vehicle, carrier, medium for the active ingredient, a preservative, cryoprotectant or a combination thereof in the pharmaceutical composition is about 75.0-99.9%.

In certain embodiments, the therapeutic agent for treating an eye disease is a small molecule (e.g., an anti-inflammatory drug such as corticosteroid or a small molecule that interferes with the interaction between VEGF or PDGF and its cognate receptor) or a nucleic acid (e.g., a nuclei acid binding to VEGF or PDGF). In on embodiment, the therapeutic agent for treating an eye disease is a receptor tyrosine kinase inhibitor for treating an eye disease. In other embodiments, the receptor tyrosine kinase inhibitor includes, but not limited to a vascular endothelial growth factor (VEGF) receptor tyrosine kinase inhibitor or a platelet-derived growth factor (PDGF) receptor tyrosine kinase inhibitor. Non-limiting examples of the receptor tyrosine kinase inhibitor include sunitinib, nintedanib, axitinib, imatinib, lenvatinib, sorafenib, vandetanib, and regorafenib. The ophthalmic pharmaceutical composition of the present invention prolongs the half-life and maintains the therapeutic concentration of the therapeutic agent at the target site, hence, sustains the therapeutic effect and reduces the frequency of drug administration.

In one aspect, the sustained release profile of the claimed ophthalmic pharmaceutical composition is due to the high drug (or therapeutic agent) encapsulation efficiency. The encapsulation efficiency of the pharmaceutical composition is at least 50%, 55%, 60%, 65%, 70%, 75%, 80%, 85% or 90%.

In another aspect, the sustained release profile of the ophthalmic pharmaceutical composition is due to the higher drug (or therapeutic agent) to lipid molar ratio. In an exemplary embodiment, the molar ratio of the therapeutic agent for treating an eye disease to the one or more lipids is above or equal to 0.20, 0.25, 0.3, 0.35, alternatively from 0.2 to 10, from 0.2 to 5, from 0.2 to 3, from 0.2 to 2.5, 0.3 to 10, from 0.3 to 5, from 0.3 to 3, from 0.3 to 2.5, from 0.35 to 10, from 0.35 to 5, from 0.35 to 3 or from 0.35 to 2.5,.

In yet another aspect, the half-life of the therapeutic agent for treating an eye disease is extended by at least 2-fold, at least 5-fold, at least 7.5-fold, at least 10-fold, or at least 20-fold in the vitreous humor compared to that of the free therapeutic agent for treating the eye disease.

The invention also provides methods of treating an eye disease, comprising the administration of an effective amount of the sustained release ophthalmic pharmaceutical composition as described herein to a subject in need thereof, whereby the symptoms and/or signs of the eye disease in the subject are reduced. Non-limiting examples of the eye disease include AMD and diabetic eye disease.

In one aspect of the invention, the sustained release ophthalmic pharmaceutical composition is formulated for injection, such as intravitreal injection, suprachoroidal administration, sub-retinal administration or periocular administration. The sustained release ophthalmic pharmaceutical composition is also formulated as eye drop or ointment for topical administration.

The dosage of the sustained release ophthalmic pharmaceutical composition of the present invention can be determined by the skilled person in the art according to the embodiments. Unit doses or multiple dose forms are contemplated, each offering advantages in certain clinical settings. According to the present invention, the actual amount of the sustained release ophthalmic pharmaceutical composition to be administered can vary in accordance with the age, weight, condition of the subject to be treated, any existing medical conditions, and on the discretion of medical professionals.

In one embodiment, the sustained release ophthalmic pharmaceutical compositions disclosed herein display a significant extended-release profile of the therapeutic agent for treating an eye disease. For example, the therapeutic agent is released from the sustained release ophthalmic pharmaceutical composition at a decelerated or slower rate, so the therapeutic concentration of the therapeutic agent is maintained over a prolonged period of time at the target site, such as the vitreous humor, for at least 168 hours. The sustained release ophthalmic pharmaceutical compositions are developed to reduce the dosing frequency to weekly, once every two weeks, once a month, once every two months, once every three months, once every four months, once every five months or once every six months.

### EXAMPLES

Embodiments of the present invention are illustrated by the following examples, which are not to be construed in any way as imposing limitations upon the scope thereof. On the contrary, it is to be clearly understood that resort may be subject to various other embodiments, modifications, and equivalents thereof, which, after reading the description herein, may suggest themselves to those skilled in the art without departing from the spirit of the invention. During the studies described in the following examples, conventional procedures were followed, unless otherwise stated.

### Example 1

### Preparation of Empty Liposome Containing Trapping Agent

Empty liposomes were prepared by a lipid film hydration-extrusion method or a solvent-injection method. For the lipid film hydration method, bilayer membrane components (e.g., DOPC/cholesterol at mole percent of 66.7/33.3) were dissolved in an organic solvent, for example, chloroform and dichloromethane. A thin lipid film was formed by removing the organic solvent under vacuum in a rotary evaporator. The dry lipid was hydrated in a trapping agent, 300 mM ammonium sulfate (AS), for 30 min at the temperature above the transition temperature to form the MLVs. Other trapping agents, such as ammonium phosphate (AP) or triethylammonium sucrose octasulfate (TEA-SOS), were also used. For the solvent injection method, bilayer membrane components (DOPC/cholesterol at mole percent of 66.7/33.3) were dissolved in an organic solvent and then injected into a stirring aqueous solution containing a trapping agent to form the MLVs. After extrusion, unencapsulated trapping agent was removed by dialysis method or diafiltration method against 9.4% sucrose solution or 0.9% NaCl to create a polyatomic ion gradient between the inner aqueous phase and the outer aqueous phase of the empty liposomes.

### Example 2

### Preparation of Liposomal Sunitinib Formulation

A reaction mixture containing 10.0 mg/mL of sunitinib (LC Laboratories, USA), empty liposomes (with 20.0 mM of lipids prepared according to Example 1), and 40 mM histidine buffer (pH 7) was incubated at 40° C. for 15 min The unencapsulated sunitinib of the reaction mixture was removed by a Sephadex™ G-50 Fine gel (GE Healthcare, USA) or dialysis bag (Spectrum Labs, USA) against a 9.4% sucrose solution to obtain a liposomal sunitinib formulation. The encapsulated sunitinib concentration and the lipid concentration of the liposomal sunitinib formulation were measured using an ultraviolet/visible (UV/Vis) spectrophotometer to calculate the drug to lipid molar ratio (D/L) of the liposomal sunitinib formulation.

The encapsulation efficiency was calculated by comparing the drug to lipid molar ratio (D/L) of the liposomal sunitinib formulation to the nominal D/L of the reaction mixture, which is dividing the initial added concentration of sunitinib by the initial added concentration of lipid of empty liposome. The particle size distribution was measured by a dynamic light scattering instrument (Zetasizer Nano-ZS90, Malvern, USA).

Using 300 mM AS as a trapping agent, the liposomal sunitinib formulation has a final D/L of 1.18, an encapsulation efficiency of 94.0%, and the mean diameter of the liposomes was 186.9 nm.

### Example 3

### Preparation of Liposomal Sunitinib with Various Lipid Compositions

The empty liposomes composed by various bilayer membranes and various trapping agents were prepared according the methods mentioned in Example 1. An initial loading concentration of 4.0 mg/mL of sunitinib or sunitinib malate was mixed with the empty liposomes according to the procedures of Example 2. Table 1 shows the drug loading profiles of liposomes with different bilayer membranes and trapping agents.

TABLE 1
The drug loading profiles of ophthalmic pharmaceutical compositions
with different bilayer membranes and trapping agents

| Bilayer | | Purified | | Average |
|---|---|---|---|---|
| membranes | | D/L | | Particle |
| (mole | Trapping | (mole/ | EE | Size |
| percent) | Agent | mole) | (%) | (nm) |
| HSPC/DSPE-PEG2000/ | 300 mM AS | 2.22 | 98.3 | n.d. |
| cholesterol | | | | |
| (59.5/0.9/39.6) | | | | |
| DOPC/DOPG/ | 300 mM AS | 0.87 | 77.0 | n.d. |
| cholesterol | | | | |
| (60/6.7/33.3) | | | | |
| DOPC/DOPG/ | 300 mM AS | 0.83 | 73.8 | n.d. |
| cholesterol | | | | |
| (66/0.7/33.3) | | | | |
| HSPC/cholesterol | 75 mM TEA-SOS | 1.02 | 81.4 | 172.9 |
| (60/40) | | | | |
| DPPC/DSPE-PEG2000/ | 200 mM AP | 0.82 | 82.0 | 170.1 |
| cholesterol | | | | |
| (66.4/0.7/32.9) | | | | |

EE, encapsulation efficiency; n.d., not determined.

### Example 4

### Preparation of Various Liposomal Receptor Tyrosine Kinase Inhibitor Formulations

Tyrosine kinase inhibitors used in this example included axitinib (LC Laboratories, USA) and imatinib mesylate (Sigma-Aldrich, USA). The empty liposomes were prepared according to Example 1 and the drugs were loaded according to the loading procedures in Example 2. For the axitinib loading studies, a reaction mixture contained 2 mg/mL of axitinib, empty liposomes (containing 300 mM AS) and 50 mM citrate buffer (pH 4.0) was incubated at 40° C. for 30 minutes. For the imatinib loading studies, a reaction mixture contained 2 mg/mL of imatinib mesylate, empty liposomes (containing 300 mM AS) and 20 mM histidine buffer (pH 6.5) was incubated at 25° C. for 30 minutes. Unencapsulated drug was removed by SephadexTM G-50 Fine gel (GE Healthcare, USA) to obtain a liposomal receptor tyrosine kinase inhibitor formulation. The D/L ratio of the liposomal receptor tyrosine kinase inhibitor formulation was calculated according to the steps in Example 2. Table 2 shows the drug loading profiles of liposomes with different bilayer membranes and receptor tyrosine kinase inhibitors.

TABLE 2
The drug loading profile of different
receptor tyrosine kinase inhibitors

| | Receptor | | Purified | |
|---|---|---|---|---|
| | Tyrosine | | D/PL | |
| Bilayer membranes | Kinase | Trapping | (mole/ | EE |
| (mole percent) | Inhibitor | Agent | mole) | (%) |
| POPC/cholesterol (66.7/33.3) | Axitinib | 300 mM AS | 0.37 | 71.0 |
| DOPC/cholesterol (66.7/33.3) | Axitinib | 300 mM AS | 0.81 | 78.5 |
| POPC/cholesterol (66.7/33.3) | Imatinib | 300 mM AS | 0.90 | 66.6 |
| DOPC/cholesterol (66.7/33.3) | Imatinib | 300 mM AS | 1.24 | 91.3 |

EE, encapsulation efficiency.

Example 5. Prolonged Release Profile of Liposomal Sunitinib Formulation

To set up the in vitro release system, (a) 50 μL of free sunitinib, (b) 50 μL of liposomal sunitinib formulation A prepared according to Example 2 (bilayer membranes composed of DOPC/cholesterol=66.7/33.3 and 300 mM of AS) and (c) 50 μL of liposomal sunitinib formulation B prepared according to Example 3 (bilayer membranes composed of HSPC/cholesterol=60/40 and 75 mM of TEA-SOS) were placed in separate dialysis bags. Each dialysis bag contained 950 μL of rabbit vitreous humor (Pel-Freez Biologicals, USA) and both ends of the dialysis bags were then sealed. Each dialysis bag was immersed in 25 mL of PBS at pH 7.4 in a 50-mL centrifuge tube and incubated in a water bath at 37±1° C. for 24 hours. At designated time points after incubation (1, 2, 4, 6, 24, 48, 122, 146 and 168 hours), 0.5 mL aliquot from the 25 mL PBS inside each centrifuge tube was sampled and 0.5 mL of fresh PBS was added to replenish the sampled aliquot. Drug concentrations of the sampled aliquots at each time point were analyzed using high performance liquid chromatography (HPLC) to create the in vitro release profile of the liposomal composition.

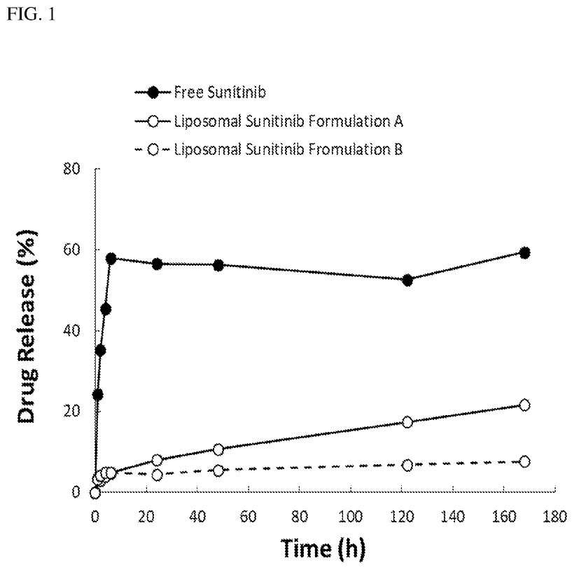

Referring to FIG. 1, sunitinib was released from the free sunitinib formulation through the dialysis bag immediately and reached a plateau after 6 hours, whereas less than 20% of sunitinib was released from the liposomal sunitinib formulation A through the dialysis bag over a 168-hour period and less than 10% of sunitinib was released from the liposomal sunitinib formulation B through the dialysis bag over a 168-hour period.

1. A sustained release ophthalmic pharmaceutical composition, comprising
(a) at least one liposome comprising a bilayer membrane, said bilayer membrane comprises at least one lipid;
(b) a trapping agent; and
(c) a therapeutic agent for treating an eye disease, wherein the molar ratio of the therapeutic agent to the lipid is equal to or higher than 0.2.

2. The sustained release ophthalmic pharmaceutical composition of claim 1, wherein the mean particle size of the liposome is from about 50 nm to 500 nm.

3. The sustained release ophthalmic pharmaceutical composition of claim 1, wherein the bilayer membrane further comprises cholesterol.

4. The sustained release ophthalmic pharmaceutical composition of claim 3, wherein the mole percentage of the cholesterol in the bilayer membrane is about 15 to about 55%.

5. The sustained release ophthalmic pharmaceutical composition of claim 1, wherein the trapping agent is selected from the group consisting of triethylammonium sucrose octasulfate, ammonium sulfate, ammonium phosphate and a combination thereof.

6. The sustained release ophthalmic pharmaceutical composition of claim 5, wherein the concentration of triethylammonium sucrose octasulfate is about 10 to 200 mM.

7. The sustained release ophthalmic pharmaceutical composition of claim 5, wherein the concentration of ammonium sulfate is about 100 to 600 mM.

8. The sustained release ophthalmic pharmaceutical composition of claim 5, wherein the concentration of ammonium phosphate is about 100 to 600 mM.

9. The sustained release ophthalmic pharmaceutical composition of claim 1, wherein the therapeutic agent for treating an eye disease is a receptor tyrosine kinase inhibitor.

10. The sustained release ophthalmic pharmaceutical composition of claim 9, wherein the receptor tyrosine kinase inhibitor is selected from the group consisting essentially of sunitinib, nintedanib, axitinib, imatinib, lenvatinib, sorafenib, vandetanib, regorafenib and a combination thereof.

11. The sustained release ophthalmic pharmaceutical composition of claim 1, wherein the therapeutic agent for treating an eye disease is encapsulated in the liposome with an encapsulation efficiency higher than about 50%.

12. A method for treating an eye disease, comprising:
administering a sustained release ophthalmic pharmaceutical composition to a subject in need thereof, said ophthalmic pharmaceutical composition comprising:
(a) at least one liposome comprising a bilayer membrane, said bilayer membrane comprises at least one lipid;
(b) a trapping agent; and
(c) a therapeutic agent for treating an eye disease, wherein the molar ratio of the therapeutic agent to the lipid is equal to or higher than 0.2.

13. The method of claim 12, wherein the half-life of the therapeutic agent in the vitreous humor of the subject is extended by at least 2-fold, at least 5-fold, at least 7.5-fold, at least 10-fold, or at least 20-fold compared to that of the free therapeutic agent in the vitreous humor of the subject.

14. The method of claim 12, wherein the sustained release ophthalmic pharmaceutical composition is administered at least once every week, at least once every two weeks, at least once a month or at least once every three months.

15. The method of claim 12, wherein the sustained release ophthalmic pharmaceutical composition is administered by injection or topical administration.

16. The method of claim 15, wherein the injection includes intravitreal administration, suprachoroidal administration, sub-retinal administration or periocular administration.

17. The method of claim 15, wherein the topical administration is by eye drop or ointment.

18. The method of claim 12, wherein the eye disease is age-related macular degeneration or diabetic eye disease.
